(19) United States
(12) Patent Application Publication
(10) Pub. No.: US 2023/0063600 A1
(43) Pub. Date: Mar. 2, 2023

(54) APPARATUS FOR SEALING AND COUPLING MATERIAL TO A BODY

(71) Applicant: Smiler Girl, LLC, Dover, DE (US)

(72) Inventors: Talli Holmes, Media, PA (US); Michael Holmes, Media, PA (US)

(21) Appl. No.: 17/899,239

(22) Filed: Aug. 30, 2022

Related U.S. Application Data
(60) Provisional application No. 63/313,319, filed on Feb. 24, 2022.
(60) Provisional application No. 63/239,052, filed on Aug. 31, 2021.

(51) Int. Cl. A61H 35/00; A61F 13/14; A61F 13/00; A61F 7/00

(52) U.S. Cl. CPC A61H 35/002; A61F 13/14; A61F 13/00059; A61F 7/00; A61F 2013/00919; A61H 2201/165; A61F 2007/0059; A61F 2007/0021; A61F 2013/00655

(57) ABSTRACT

A bandage-type apparatus includes a skin condition compartment that is adhered to cover a skin condition with leak-proof adhesive surrounding the compartment that attaches to skin adjacent the skin condition and with a non-adhesive material of the compartment covering the skin condition. A liquid pouch or container with a releasable liquid is coupled to the skin condition compartment and configured for the liquid, such as saline solution, to be released into the compartment to provide a bath or soak in the leak-proof, body-adhered skin condition compartment to provide treatment or relief to the skin condition, such as a sore nipple of a breastfeeding mother.

### CROSS-REFERENCE TO RELATED APPLICATIONS

This application claims the benefit of priority of provisional application No. 63/239,052 filed Aug. 31, 2021 and provisional application No. 63/313,319 filed Feb. 24, 2022, all of which are incorporated herein by reference.

### BACKGROUND OF THE INVENTION

The present invention relates to a bandage-type apparatus for applying liquids, such as saline solution or medicinal fluids, to a wound, skin condition or other target region of the body that would benefit from a bath or soak. For example, nursing mothers often experience nipple rawness, wounds, and soreness injuries for which conventional treatments include holding cups or bowls of saline solutions in an uncomfortable and mobility-limited manner to the irritated nipples for repeated intervals of several minutes throughout a day. During such treatments, the mother is generally unable to provide care to their infant or engage in any other activities since she must remain stationary with the cups or bowls held or supported against the nipples. Additionally, preparation of the saline solution and filling and cleaning of the cups or bowls adds inconvenience and additional time taken away from the busy mother.

Accordingly, a need exists for an easy and quick-to-apply bath or soak solution, such as saline, to adhere over a body wound, like nipples with nursing injuries and soreness, while the user can remain mobile and carry out daily activities.

### SUMMARY OF THE INVENTION

To answer this need, in embodiments of the invention a leak-proof wound or skin condition compartment (adhesive-free region that holds liquid over wound) is provided to receive and/or cover a wound, skin condition, injury or other portion of the body that would benefit from a liquid bath or soak. A seal, such as a liquid-proof or waterproof adhesive surrounding the perimeter of the skin condition compartment to stick to skin around the target skin condition, is provided that holds the liquid bath or soak in the apparatus and allows the bandage-type apparatus to remain in place and hold the treating liquid, such as saline solution or medicinal solution, on the body while the user can remain mobile and carry out normal activities. In some embodiment a liquid-proof silicone seal may be provided surround the skin condition compartment and help hold the soak or bath solution in the compartment for treating the wound.

In embodiments of the invention, the adhesive seal surrounding and adhering the leak-proof skin condition compartment may include a removal backing that blocks the adhesive from sticking to objects until desirable for adhering to the body location. This could include “peel away” paper backings such as used on conventional bandages, stickers and body tapes which permit one-step removal of the backing on the adhesive of the adherable skin condition compartment at the time of attachment to the skin and body.

In some embodiments of the invention one or more liquid-filled pouches are coupled to the skin condition compartment from which the liquid can be released into the compartment after the use has adhered the compartment with the leakproof adhesive seal over the wound (e.g. nipple and areola). The one or more pouches may be connected to the skin condition compartment by any known mechanisms for releasably containing a liquid, such as a press-seal, frangible seal, valve, tear-away lining, and the like. The user would adhere the compartment at and over the desired skin condition/body location and then open the seal(s) in such manner determined by the type of seal or valve so that the liquid solution, like saline solution, is released from the pouch(es) and held as a bath or soak over the wound. In some embodiments, the one or more pouches may be positioned and coupled above the compartment so that gravity helps cause the liquid to flow down into the compartment and soak or bath the wound area, such as nipple and areola of a breastfeeding mother.

In other embodiments of the invention, a bandage-type apparatus with a leak-proof skin condition compartment may include a sealable opening or valve into which a liquid solution may be dispensed or injected. In some embodiment the liquid solution might be packaged with pre-measured amounts of liquid, like saline, antiseptic, soap and water, or medicinal solution, and squeezed or injected from a tube, bottle, packet, or similar containers that preferably couple to opening or liquid insertion location on the skin condition compartment. Once the liquid has been dispensed into the covering compartment, the opening, valve, or similar insertion location is closed to retain the bath or soak solution in the compartment and on the wound site.

In various embodiments of the invention, a bandage-type apparatus that adheres with a leakproof compartment over a skin condition via adhesive to skin surrounding the condition and holds a liquid solution, such as a saline, antibacterial, body cleaning solution, moisturizer or medicinal solution, may provide a bath or soak for nipples that are sore and injured from injury, locations of surgery on the body, piercing sites on the body, insect bites, diseased tissue (such as cancer), infections, injuries and wounds to the skin, scars, burns, sunburns, tattoos or tattoo removals and like skin conditions where a soak or bath would benefit the skin and tissue. In some embodiments, a device of the present invention may be adhered not only to humans, but also to animals that could benefit from coupling materials to a condition or part of the animal body.

In various embodiments, devices of the invention can attach over body locations other than skin conditions or wounds, such as an eye or eyes where materials are sealed against the eye area. In some embodiments, a device of the invention may be adhered to a larger body part or appendage of a human or animal to couple materials to that body part. For example, a baby or person may cut a finger or hand and a device of the present invention may be shaped and sized to contain the whole finger or hand and materials may be coupled to treat the body part or the condition of the body part by providing a compartment as a “sealed” bubble over that part.

In embodiments of the invention, the liquid to be provided in the skin condition covering compartment may also be adjusted to a preferred temperature, such as with heating or cooling as desirable for administering the skin condition soak.

In other embodiments of the invention, a dressing or gauze might also be pre-soaked or soaked with liquid before application to a wound and held in a place by an adhesive skin condition compartment with a surrounding leak-proof adhesive adhering to surrounding skin as disclosed by the present invention. A user could, for instance, place a saline-soaked dressing into a leak-proof skin condition compartment and then the compartment could be adhered over the wound location and hold the “wet” dressing in place (via leak-proof adhesive attaching to skin around the wound) while the user is able to remain mobile.

In some embodiments, a compartment or enclosure device of the present invention may adhere cosmetic, moisturizing, or other non-medicinal materials against desire desired parts of the body.

### BRIEF DESCRIPTION OF THE DRAWINGS

FIG. 1 is a schematic diagram of a nipple covering apparatus with releasable saline in pouches coupled to a nipple covering compartment having a leak-proof adhesive seal around the compartment and in the shape of a bear in an embodiment of the invention.

FIG. 2A is a schematic diagram of a nipple covering apparatus with releasable saline in a pouch with a releasable valve coupled to a nipple covering compartment having a leak-proof adhesive seal around the compartment and in the shape of a pear in an embodiment of the invention.

FIG. 2B is a schematic diagram of a nipple covering apparatus with releasable saline in a pouch with a releasable valve (opened by pulling on string to leaf) coupled to a nipple covering compartment having a leak-proof adhesive seal with peel-away backing paper and in the shape of a pear in an embodiment of the invention.

FIG. 3 is a perspective view illustrating a reusable silicone apparatus with a domed substance holding compartment having a surrounding seal perimeter, such as for receiving adhesive, that includes a substance delivery tube and a syringe for providing substance into the apparatus for treatment of wound, injury and/or skin and body tissue beneath the aperture of the dome surrounded by the adhesive that adheres to the body in an embodiment of the invention.

FIG. 4 is a schematic diagram of a transfer adhesive ring that may be to adhere the apparatus shown in FIG. 3 to a body in an embodiment of the invention.

### DETAILED DESCRIPTION OF THE INVENTION

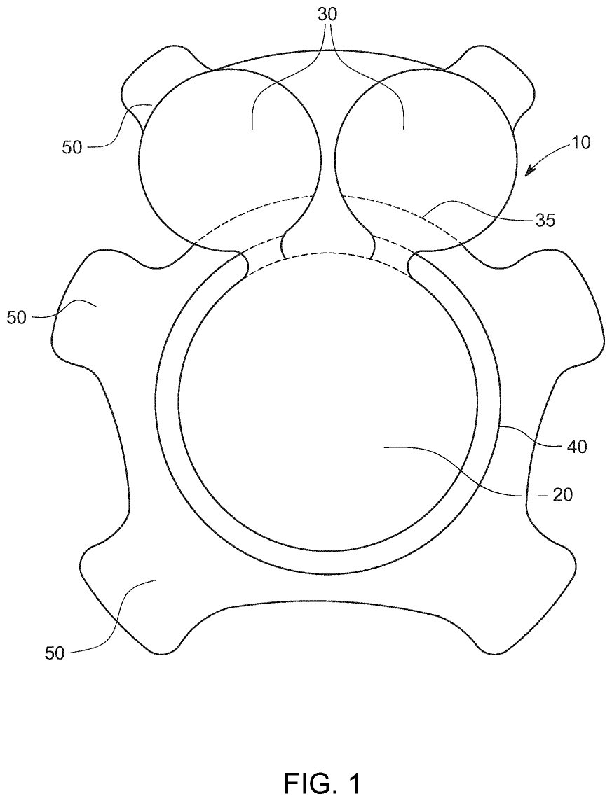

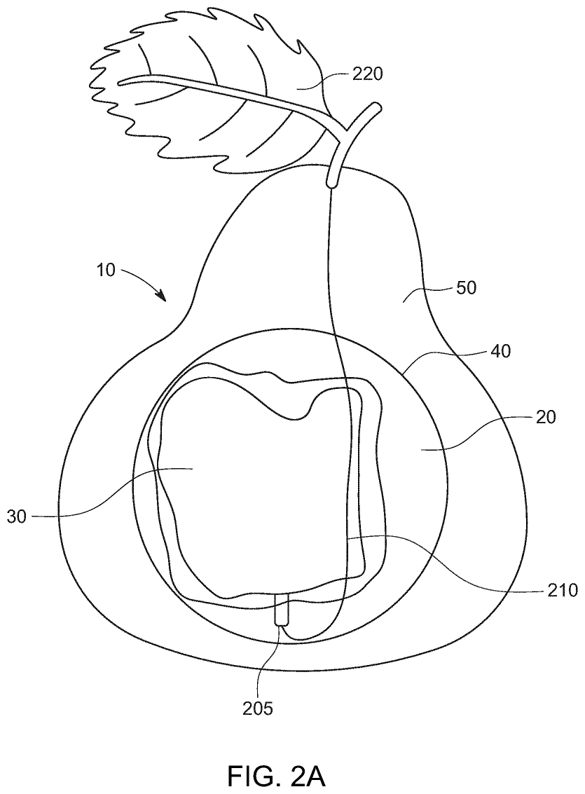

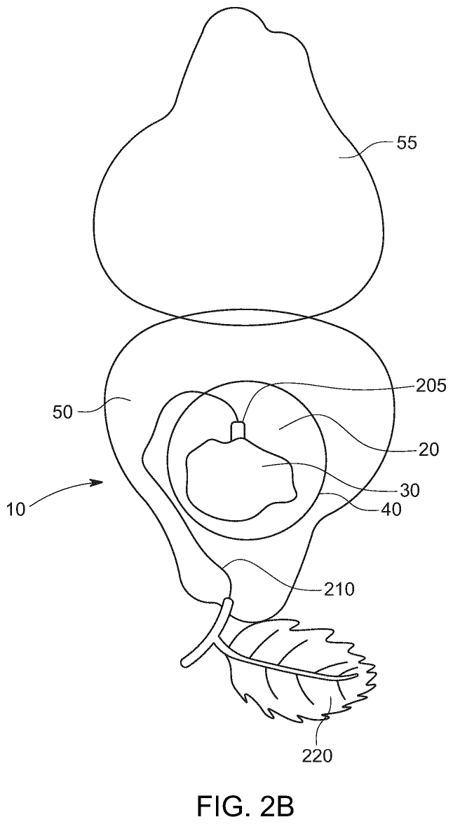

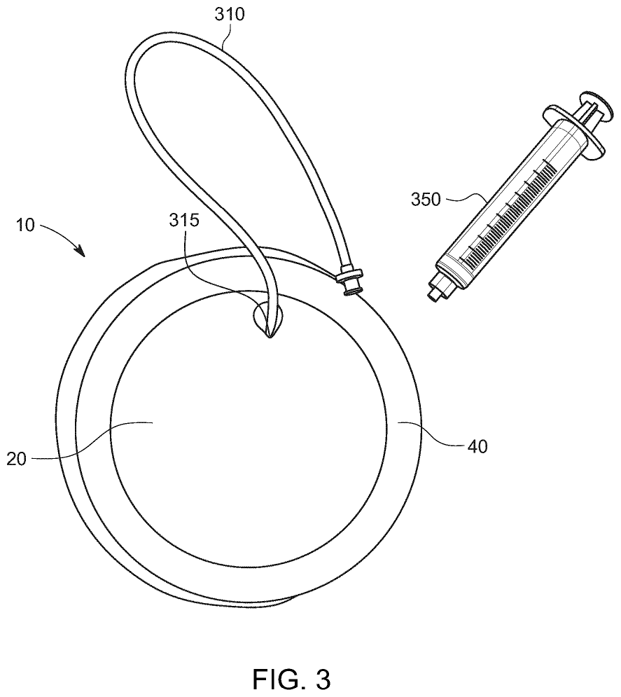

FIGS. 1-4 illustrate and are described with respect to specific embodiments of the invention. In the broadest sense, the invention is directed to a treatment device 10 attachable for applying and retaining a treatment substance at a location on a human or animal body. The treatment device 10 includes substance holding compartment 20 that has a leak-proof seal 40 around the perimeter of compartment 20. The compartment 20 receives a treating substance (not shown), such as a from a substance storage component 30 (e.g. pouch in FIGS. 1, 2A and 2B) or a delivery tube 310 (see FIG. 3). The substance holding compartment 20 is coupled to a body-compatible adhesive or attachment component 50 that secures the substance holding compartment 20 over the body target area and the desirable substance is delivered into the compartment 20 and held in place to on the body target area. In embodiments, the preferred body target area is a nipple (such as needing treatment due to breast-feeding injury) and the preferred substance is saline solution.

Referring to FIG. 1, a schematic diagram of a nipple covering apparatus 10 with releasable saline in pouches 30 (near bear eyes) coupled to a nipple and areola covering compartment 20 (bear belly) having a leak-proof seal 40 around the compartment and in the shape of a bear or similar animal, object, character, flower, food, plant, and the like. The pouches 30 may include press seal, frangible seal, “burst” seal, valves, or similar-type of liquid-releasing mechanism 35 so that liquid enters the skin condition compartment 20 after the user attaches the adhesive/attachment region 50 and compartment 20 (itself adhesive-free) at the wound location when a bath/soak is desired for application.

The adhesive region 50 around the skin condition compartment 20 and leak-proof seal 40 of the apparatus 10 may include a waterproof adhesive tape or bandage, like what is shown at the website: https://spidertech.com/us/category-pre-cuts (incorporated herein by reference). Similar leak-proof adhesive that couples to the skin condition compartment 20 and can attach to surrounding skin around the skin condition that is covered may also include adhesive material like the “Seal-Tight Shield and Shower Patch” disclosed at https://www.amazon.com/Shied-Dressing-Protector-Watertight-Protection/dp/B001G0N6HW (incorporated herein by reference).

In other embodiments, other mechanisms as an alternative to adhesive connection may be used to provide a sealed connection of the apparatus 10 with the skin compartment 20 or aperture having a leak-proof seal 40 perimeter to the desired body target area. For example, a sealed connection of devices of the invention could be provided by a harness or similar attachment or coupling device using magnets, Velcro (hook and loop material), and click-in or lock-in compartments or cups. In other embodiments, skin glue or similar adhering chemicals that provides a leakproof seal 40 may couple between the skin and apparatus to hold the treatment device in sealed position with the compartment 20 over the body condition being treated.

A bandage-type apparatus in embodiments of the invention may be provided in different shapes and sizes with an aesthetic design like a bear (or other animals, characters, objects (see FIGS. 2A and 2B showing a pear)), with a donut-like hole cut in the center to allow the nipple (or other affected areas) to be placed in the compartment 20. A liquid (or gel, medicament, ointment, or other substance desired for application to the body) can be provided in a pre-measured/filled container or an integrated liquid-filled bag/pouch 30 (such as saline in the case of nipple care) that is coupled to the compartment 20 (adhesive-free in region covering the skin condition) that has the adhering tape/bandage adhesive material 50, so as to provide a “bath” to care for the skin condition, wound, muscle condition or other body target area benefitting from a retained substance sealed over that body area. When the liquid or substance enters the compartment 20 it is held in place by the waterproof/leak-proof seal 40 coupled to adhering tape 50 or bandage to the skin surrounding the compartment 50. In some embodiments, the tape/bandage's inner-circle/lining of the compartment includes a silicone-based ring attached to it, to act as a leak-proof seal 40 exhibiting an impermeable barrier to prevent the liquid/substance from seeping through the adhesive attachment mechanism 50. The compartment 20 could be made of a soft material in some embodiments and coupled to a bag/cup in a press-seal manner similar to a Ziploc storage bag. In other embodiments, the compartment 20 intended to retain a substance for treating the body can be provided as a harder disc that could allow for the bag as the substance storage component 30 to “attach” or “click” into it, with a twist. The substance would then be delivered from the bag storage component 30 to the disc compartment 20.

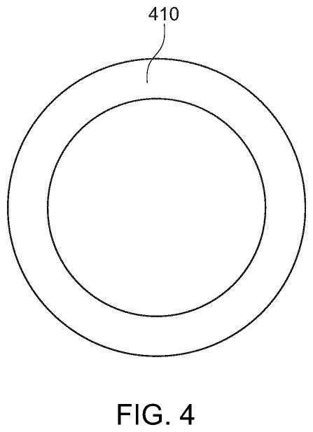

In different embodiments of the invention, liquid may be provided to the skin condition compartment in a variety of ways, such as, for example: 1) an injection or substance entry port 315 (preferably that is closable) into the substance holding compartment 20 of the bandage, allowing for self-filling to the desired volume (see e.g. FIG. 3), such as with a substance delivery tube 310 and syringe 350—and in some embodiments may include transfer adhesive rings 410 (including product nos. 1522 and 1524 from 3M Company (St. Paul, Minn.)) (see FIG. 4) that adhere to the seal area 40 of the device of FIG. 3; 2) saline or liquid-filled pouches 30 already attached to the compartment 20 of the tape/bandage (like near the “eyes” of the this bear-shaped form of FIG. 1, with ready-made substance release mechanism 35 (e.g. perforations, press seal, valves and the like) for after the bandage is attached and the person is ready to fill compartment 20), 3) a “tear-away” lining or a valve 205 that opens a pre-filled saline bag 30 in or coupled to the compartment 20 that would allow the compartment 20 to be adhered over the skin condition, and then the individual can pull a tab 220 attached by a string 210 or similar mechanism to the lining or valve 205 that allows for the inner lining to get stripped away or a valve 205 to open (such as shown in FIGS. 2A and 2B) where the stem could act as the tab 220 connected to the string 210 that is connected for removing the lining or opening of the valve 205 to release the liquid/substance and create a soak or bath on the skin condition in the compartment 20. With respect to the embodiment of FIGS. 2A and 2B, and in other similar embodiments, the adhesive/attachment mechanism 50 may include a backing 55 that is peeled off to allow for adhering the substance compartment 20 coupled to the attachment mechanism 50 to the body similar to adhesive bandages in which a backing paper is first removed to adhere the bandage over a wound.

In embodiments of the invention, the substance to be provided in the skin condition covering compartment 20 may also be adjusted to a preferred temperature, such as with heating or cooling as desirable for administering the skin condition soak. For example, it is often desirable when applying an apparatus of the invention for soaking a nipple and areola of breastfeeding mother to use warm saline as the liquid. Accordingly, in various embodiments of the invention the liquid in a container or pouch before providing to the skin condition compartment might be heated to a desired temperature in a microwave, steamer, in hot or warm water, in running hot or warm water, and the like. In other embodiments of the invention, it may be desirable to cool the liquid in a container or pouch before providing to the skin condition compartment such as by refrigeration, in a freezer, in cold water, in running cold water, and the like.

In other embodiments of the invention, alternatives to liquids may be introduced into compartment or enclosure treatment devices of the present invention and sealed against the treatment target of the body for desired treatments, including solids (including ice and frozen materials and/or materials melting with body temperature), gases, gels, emulsions, colloids, medicaments, ointments, creams and super-fluids. In some embodiments combinations of different types of materials—e.g. liquid and solid together—may be provided in the “open” compartment 20 adjacent the body. Depending on the treatment material(s) desired to be introduced in devices of the invention, different sizes and shapes of compartments and material introduction openings with different retaining mechanisms for enclosing the material(s) against the body may be used (e.g. a closing cap or press-seal for ice, larger solids or other materials may be provided at the material introduction opening of the compartment or enclosure). It will be appreciated that such devices having a leak-proof seal will help contain water from melting ice within the compartment and over the treatment area without spreading water over other parts of the body or on to clothing.

In some embodiments, a flexible and cushioned exterior is advantageous for the user of the apparatus. The exterior of the apparatus could include wet suit type of material that would allow the full form of the user to move with activity and be able to be knocked or bumped or poked and still come away intact. In further embodiments, the skin/body condition compartment would have a relatively solid inner form and material, such as silicone and the like, that would keep, in the case of a woman's breast, the nipple (and areola) from coming in contact (and being irritated) with a pad material and/or bandage material of the apparatus.

The outer exterior layer of the skin condition compartment might include a wet suit-type material that would perform like sports tape (in the “first layer”) previously described as advantageous for adhering to the skin for attaching the skin condition compartment. The attachment of the exterior layer to the underlying first layer would require a strong adhesive, but could also be secured secondarily with snaps at the top (perhaps visually appearing as the bear's eyes). The information at the incorporated Spider Tech information link shows tape with the strength to support a pregnant woman's belly and the incorporated Seal-Tight Shield information link shows adhesive patch that holds up and remains leaf-proof in a shower.

In embodiments of the invention, a bandage-type apparatus with a liquid-fillable and leakproof compartment is applied to a woman's breast, such as in the case of soreness due to breastfeeding, or after breast surgery, or for use while bathing or showering to avoid sensitivity/pain. In other embodiments of the invention, the bandage-type apparatus may be modified in shape to permit bathing or soaking of other areas of the body but perform similarly in function. This could include many types of skin conditions, such as treating wounds, injuries, cuts, scrapes, burns, sunburn, dry skin, infections, sensitive skin, bruises, ulcers, sores, insect bites, diseased tissue, piercings, tattoos, scars, and surgical sites on the body. Depending on the desired bath or soak, a variety of liquid and fluid solutions might be adapted for use with embodiment of the invention, including saline, soap, and water (in the case of a bath), medicinal solutions, antibiotics, scar treatments, moisturizers, cosmetics, aloe and/or other healing/cooling agents or chemicals to treat burns or damaged skin. In other embodiments, a dressing or gauze might also be pre-soaked or soaked with liquid before application to a wound and held in a place by an adhesive skin condition compartment with a surrounding adhesive layer coupled to the compartment (that retains the pre-soaked material over the skin condition) as disclosed by the present invention. A user could, for instance, place a saline-soaked dressing into a leak-proof skin condition compartment with an adhesive tape/seal around the periphery and then the compartment could be adhered the wound location and hold the “wet” dressing in place while the user is able to remain mobile.

In embodiments, components of the invention may be packaged and provided as an all-in one product or as a kit. In all-in one product, such as shown in embodiments of FIGS. 1A and 2A and 2B, may be a bandage-type apparatus that incorporates the liquid solution within pouches or similar liquid storage regions of the apparatus and a paper backing is removed by the user from the adhesive portion that surrounds the skin condition covering compartment, the apparatus is adhered with the adhesive portion to the skin of desired part of the body with the wound covered by the skin condition compartment (preferably adhesive-free) and then the user releases the liquids from the integral liquid storage region or pouch into the leak-proof compartment. In a kit, the bandage-type apparatus with skin condition compartment and adhesive portion surrounding the compartment (and paper backing before use provided over the adhesive portion) may be packaged separately with a container or multiple containers of liquid, such as a medicinal solution or saline. The liquid can then be provided into the skin condition compartment (preferably after a leak-proof adhering of the skin condition compartment (adhesive-free) over the wound and adhesive region to the skin adjacent the skin condition to be covered) through a sealable and liquid-proof opening, such as a cap, valve, injectable opening, press seal, and the like, to provide a bath or soak in the skin condition compartment over the wound or other skin condition.

Various embodiments of the invention have been described. It will, however, be evident that various modifications and changes may be made thereto, and additional embodiments may be implemented, without departing from the broader scope of the invention as set forth by the claims. This specification is to be regarded in an illustrative rather than a restrictive sense.

What is claimed is:

1. An apparatus comprising:
a leak-proof skin condition compartment having an outer surface and opposite inner surface with a non-adhesive region on the inner surface;
a leak-proof adhesive region coupled to the skin condition compartment; and
a liquid-filled pouch including a releasable liquid and coupled to the skin condition compartment.

2. The apparatus of claim 1, wherein the liquid is saline.

3. The apparatus of claim wherein the liquid is a medicinal solution.

4. The apparatus of claim 1, wherein the liquid-filled pouch includes at least one of a press seal, frangible seal, and valve.

5. The apparatus of claim 1, wherein the apparatus is shaped in an aesthetic design of an animal, character, food, flower or plant.

6. The apparatus of claim 9, wherein the apparatus is shaped as a bear.

7. The apparatus of claim 9, wherein the apparatus is shaped as a pear.

8. A method of treating a skin condition comprising adhering the skin condition compartment of claim 1 over the skin condition with the leak-proof adhesive region coupled to skin adjacent the skin and with the non-adhesive region over the skin condition and releasing the liquid into the skin condition compartment to provide a soak or bath to the skin condition.

9. The method of claim 8, wherein the skin condition is a on a breast.

10. The method of claim 9, wherein the non-adhesive region of the skin condition compartment is applied over a nipple and areola.

11. The method of claim 10, wherein the skin condition is a wound caused by breastfeeding.

12. The method of claim 8, wherein the skin condition is selected from the group consisting of a wound, injury, cut, scrape, bruise, burn, sunburn, dry skin, infection, sensitive skin, insect bite, diseased tissue, piercing, tattoo, tattoo removal, scar, and surgical site.

13. The method of claim 12, further comprising heating or cooling the liquid to a desired temperature before releasing the liquid into the skin condition compartment.

14. An apparatus comprising:
a leak-proof compartment having an outer surface and opposite inner surface with a non-adhesive region on the inner surface;
a body attachment mechanism including skin-compatible adhesive coupled to the leak-proof compartment; and
a substance-containing storage component coupled to the leak-proof component, wherein the storage component includes a seal or valve configured to be actuated to release substance from the storage component into the leak-proof compartment.

15. The apparatus of claim 14, wherein the substance is a liquid.

16. The apparatus of claim 15, wherein the liquid includes saline.

17. The apparatus of claim 16, wherein the leak-proof compartment is configured to receive and bathe a nipple.

18. The apparatus of claim 14, wherein the skin-compatible adhesive is releasably coupled to a removable backing.

19. The apparatus of claim 15, wherein the substance is selected from the group consisting of a liquid, gas, gel, emulsion, colloids medicament, ointments cream and super-fluid.

20. The apparatus of claim 14, further comprising a silicone seal at the perimeter of the leak-proof compartment.
